(19) United States
(12) Patent Application Publication
(10) Pub. No.: US 2025/0345195 A1
(43) Pub. Date: Nov. 13, 2025

(54) IMPLANT DELIVERY SYSTEMS AND METHODS

(71) Applicant: BOSTON SCIENTIFIC SCIMED, INC., Maple Grove, MN (US)

(72) Inventors: JOHN THOMAS O'DRISCOLL, Gort (IE); THOMAS MARTIN KEATING, Galway (IE); DANIEL TUCK, Galway (IE); FIONN STAPLETON, Galway (IE); CELINE GLYNN, Galway (IE); HARRY MULLEADY, Galway (IE); GARY GILMARTIN, Foxford (IE)

(73) Assignee: BOSTON SCIENTIFIC SCIMED, INC., Maple Grove, MN (US)

(21) Appl. No.: 19/202,739

(22) Filed: May 8, 2025

Related U.S. Application Data
(60) Provisional application No. 63/644,766, filed on May 9, 2024.

(51) Int. Cl. A61F 2/95; A61F 2/04; A61M 25/00; A61M 25/09

(52) U.S. Cl. CPC A61F 2/9517; A61F 2002/041; A61F 2002/9511; A61M 25/0097; A61M 25/09

(57) ABSTRACT

A delivery system for delivering an implant to a body lumen of a patient. The delivery system may include a hub, an elongate shaft coupled with the hub, and a thread for securing the implant to the elongate shaft. The elongate shaft may include a proximal portion at least partially defining a first lumen and a second lumen, a distal portion at least partially defining the first lumen, and a side port in the proximal portion and distal of the hub. The side port may be in communication with the first lumen. The thread may include a distal portion configured to wrap around the distal portion of the elongate shaft and/or the implant and a proximal portion configured to extend through the second lumen. The delivery system may be configured to deliver the implant to a target location in a lumen of a biliary tract of the patient.

### CROSS-REFERENCE TO RELATED APPLICATIONS

The application claims the benefit of U.S. Provisional Patent Application Ser. No. 63/644,766, filed on May 9, 2024, the disclosure of which is incorporated herein by reference.

### TECHNICAL FIELD

The present disclosure relates generally to methods and apparatuses for delivery of implants. More particularly, the disclosure relates to different configurations and methods of manufacture and use of catheter systems for delivery stents.

### BACKGROUND

Implantable stents are devices that are placed in a body structure, such as a blood vessel, esophagus, trachea, biliary tract, colon, intestine, stomach or body cavity, to provide support and to maintain the structure open. Delivery systems are used to deliver stents to these locations. Stents and stent delivery systems are manufactured by any one of a variety of different manufacturing methods and may be used according to any one of a variety of methods. Of the known medical devices, delivery systems, and methods, each has certain advantages and disadvantages. There is an ongoing need to provide alternative medical devices and delivery devices as well as alternative methods for manufacturing and using medical devices and delivery devices.

### SUMMARY

This disclosure is directed to several alternative designs, materials, and methods of manufacturing medical device structures and assemblies, for delivery of implants to a target location.

In a first example, a delivery system for delivering an implant to a body lumen may include a hub, an elongate shaft coupled with the hub, the elongate shaft comprising a proximal portion at least partially defining a first lumen and a second lumen, a distal portion at least partially defining the first lumen, and a side port in the proximal portion and distal of the hub, the side port is in communication with the first lumen, and the delivery system further comprises a thread including a distal portion configured to wrap around the distal portion of the elongate shaft and a proximal portion configured to extend through the second lumen.

Alternatively or additionally to any of the examples above, the proximal portion of the elongate shaft may be formed from a first tubular structure having a first outer diameter and the distal portion of the elongate shaft may be formed from a second tubular structure having a second outer diameter smaller than the first outer diameter.

Alternatively or additionally to any of the examples above, the first outer diameter may be 3.0 millimeters or less.

Alternatively or additionally to any of the examples above, the second tubular structure may extend into the proximal portion of the elongate shaft.

Alternatively or additionally to any of the examples above, the second lumen may extend from a location proximate the hub to a distal opening proximal of a distal end of the elongate shaft.

Alternatively or additionally to any of the examples above, the side port may be a first skived port and the distal opening is a second skived port.

Alternatively or additionally to any of the examples above, the delivery system may further include a pull member coupled to a proximal end of the thread.

Alternatively or additionally to any of the examples above, the delivery system may further include a distal tip at least partially defining the first lumen and forming a distal end of the distal portion of the elongate shaft.

Alternatively or additionally to any of the examples above, the distal tip may define an opening in communication with the tip lumen.

In another example, a delivery system for delivering an implant to a body lumen, may include a hub, an elongate shaft coupled with the hub, the elongate shaft comprising a first lumen, a second lumen, a reduced diameter portion, and a side port in communication with the first lumen and located between the hub and the reduced diameter portion, an expandable implant disposed about the reduced diameter portion of the elongate shaft, and a thread including a distal portion wrapped around the expandable implant and a proximal portion configured to extend through the second lumen, wherein the thread is configured to release the expandable implant for expansion in response to pulling on the proximal portion of the thread.

Alternatively or additionally to any of the examples above, the delivery system may further include a distal tip at least partially defining the first lumen and forming a distal end of the distal portion of the elongate shaft.

Alternatively or additionally to any of the examples above, the delivery system may further include a guidewire extending through the distal tip, the first lumen, and the side port.

Alternatively or additionally to any of the examples above, the second lumen may extend from a location proximate the hub to a distal opening located proximal of the reduced diameter portion of the elongate shaft.

Alternatively or additionally to any of the examples above, the delivery system may further include a pull member coupled to a proximal end of the thread at a location proximate the hub.

Alternatively or additionally to any of the examples above, the elongate shaft may have a maximum outer diameter of 3.0 millimeters.

In another example, a method for delivering an implant to a body lumen may include advancing a delivery system to a target location in a body lumen over a guidewire extending through a first lumen and a side port of the delivery system, the delivery system comprising a hub, an elongate shaft coupled with the hub, the elongate shaft comprising the first lumen, a second lumen, a reduced diameter portion, and the side port located distal of the hub, an expandable implant disposed about the reduced diameter portion of the elongate shaft, and a thread including a distal portion wrapped around the expandable implant and a proximal portion configured to extend through the second lumen, and the method may further include applying a pulling force to the proximal portion of the thread to unravel the thread wrapped around the expandable implant and allow the expandable implant to expand.

Alternatively or additionally to any of the examples above, the target location in the body lumen may be a target location in a lumen of a biliary tract.

Alternatively or additionally to any of the examples above, the expandable implant may comprise a self-expanding stent.

Alternatively or additionally to any of the examples above, the delivery system may comprise a pull member coupled to a proximal end of the thread.

Alternatively or additionally to any of the examples above, the elongate shaft may have a maximum outer diameter of 3.0 millimeters.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The invention may be more completely understood in consideration of the following detailed description of various embodiments in connection with the accompanying drawings, in which:

FIG. 1 is a schematic side view of an illustrative implant;

FIG. 2 is a schematic side view of an illustrative delivery system for delivering an implant;

FIG. 3 is a schematic cross-section view of the illustrative delivery system depicted in FIG. 2.

FIG. 4 is a schematic diagram of an illustrative method for delivery of an implant to a target location;

FIGS. 5 and 6 schematically depict an illustrative technique for releasing an implant from a delivery system; and

FIGS. 7 and 8 schematically depict an illustrative technique for releasing an implant from a delivery system.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may be indicative as including numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

Although some suitable dimensions, ranges, and/or values pertaining to various components, features and/or specifications are disclosed, one of the skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

For purposes of this disclosure, “proximal” refers to the end closer to the device operator during use, and “distal” refers to the end further from the device operator during use.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

It is noted that references in the specification to “a configuration”, “some configurations”, “other configurations”, etc., indicate that the configuration described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with one configuration, it should be understood that such feature, structure, or characteristic may also be used in connection with other configurations whether or not explicitly described unless clearly stated to the contrary.

Stent technologies configured to produce a stent with a desired foreshortening, conformability, and migration resistance (e.g., fully or partially coated stents, stents with mechanical fixations, stents with increased diameter and/or surface areas, etc.) as well as stents including active coatings (such as, but not limited to, drugs or anti-microbials) may result in difficulties loading such stents onto standard coaxial delivery systems (e.g., over-the-wire delivery systems). Further, stents designed to achieve desired foreshortening, conformability, and migration resistance may lead to higher stent deployment forces and/or other difficulties in deploying the stent due to radially outward forces of the stent acting on a sheath covering the stent during axial removal of the sheath to release the stent. As an alternative to such sheath-based over-the-wire delivery and deployment techniques and systems, stents with desired foreshortening, conformability, and migration resistance may be loaded onto a rapid exchange catheter configured for placing such stents in small diameter vessels (e.g., the biliary tract, etc.), where the catheter allows for removing a component securing the stent on the catheter radially rather than axially. Utilizing a rapid exchange catheter with the stent secured radially on the catheter as a stent delivery system may facilitate achieving desired flexibility, strength, and pushability of the delivery system for crossing lesions and traversing vasculature when delivering the stent to a target location (e.g., a location in the biliary tract), while enabling stent delivery with a single operator maintaining a guide wire position. Further, the improved delivery system may result in a more efficient delivery of the stent to the target location in the biliary tract, which may reduce fluoroscopy times, than when over-the-wire delivery systems with axial stent securing components are utilized.

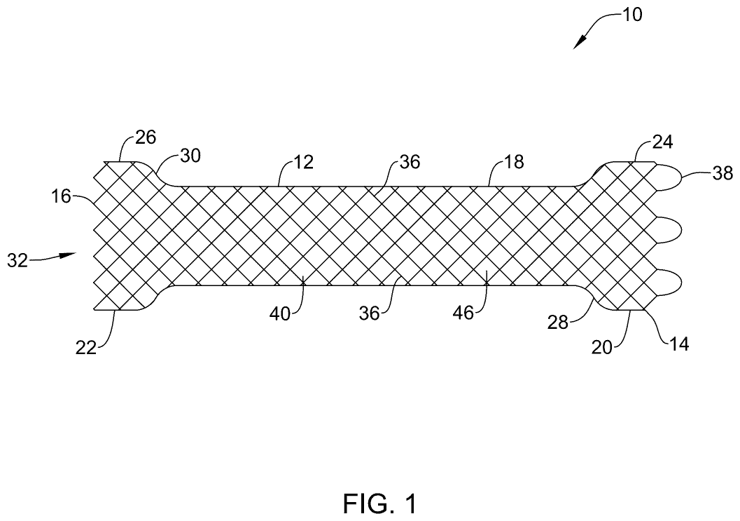

FIG. 1 illustrates a side view of an illustrative configuration of an expandable implant (e.g., an expandable endoluminal implant), such as, but not limited to, a stent 10. In some examples, the stent 10 may be formed from an elongate tubular member 12. While the stent 10 is described as generally tubular, it is contemplated that the stent 10 may take any cross-sectional shape desired. The stent 10 may have a first end 14 (e.g., a proximal end), a second end 16 (e.g., a distal end), and an intermediate region 18 disposed between the first end 14 and the second end 16. The stent 10 may include a lumen 32 extending from a first opening adjacent the first end 14 to a second opening adjacent to the second end 16 to allow for the passage of fluids, etc. (e.g., bile when the stent is placed in the biliary tract, etc.)

The stent 10 may be expandable from a first radially collapsed configuration (not explicitly shown) to a second radially expanded configuration. In some cases, the stent 10 may be deployed to a configuration between the collapsed configuration and a fully expanded configuration. The stent 10 may be structured to extend across a stricture and to apply a radially outward pressure to the stricture in a lumen to open the lumen and allow for the passage of fluids, etc.

The first end 14 of the stent 10 may include one or more loops 38. The loops 38 may be configured to receive a forceps, a retrieval tether or suture (not explicitly shown) interwoven therethrough or otherwise passing through one or more of the loops 38, and/or other suitable retrieval components. The retrieval tether or suture may be used to collapse and retrieve the stent 10, if so desired. For example, the retrieval tether or suture may be pulled like a drawstring to radially collapse the first end 14 of the stent 10 to facilitate removal of the stent 10 from a body lumen. Further, the one or more loops 38 may be configured to facilitate mitigating tissue trauma and stent migration.

The stent 10 may have any suitable structure configured to achieve a desired foreshortening, conformability, and migration resistance when implanted in a body lumen (e.g., a lumen of the biliary tract, etc.) In some cases, the stent 10 may be a self-expanding stent (SES), but other suitable configurations are contemplated. Example configurations of stent structures include, but are not limited to, a wound structure, a woven structure, a braided structure, a knitted structure, a knotted structure, a laser cut structure, and/or other suitable structure. In some examples, the structures of the stent 10 may be configured for placing the stent in a lumen of the biliary tract, but other suitable configurations are contemplated. In some examples, the stent 10 may have a woven structure fabricated from one or more filaments or struts 36 forming a tubular wall. In some examples, the stent 10 may be wound, knitted, or braided with a single wire, filament, or strut. Although other configurations are contemplated, the single strut may be interwoven with itself and define open cells 46 extending through the thickness of the tubular wall of the stent 10. In other examples, the stent 10 may be braided with a plurality of wires, filaments, or struts interwoven together and defining open cells 46 extending through the thickness of the tubular wall of the stent 10. Some illustrative stents 10 including braided filaments include, but are not limited to, the WALLFLEX®, WALLSTENT®, and POLY FLEX® stents, made and distributed by Boston Scientific, Corporation. In some examples, the stent 10 may be knitted, such as the ULTRA FLEX™ stents made by Boston Scientific Corporation. In some examples, the stent 10 may be of a knotted type, such as the PRECISION COLONIC™ stents made by Boston Scientific Corporation. In some examples, the stent 10 may be a laser cut tubular member, such as the EPIC™ stents made by Boston Scientific Corporation. A laser cut tubular member may have an open and/or closed cell geometry including one or more interconnected monolithic filaments or struts defining open cells 46 therebetween, with the open cells 46 extending through the thickness of the tubular wall.

An inner and/or outer surface of the tubular wall of the stent 10 may be entirely, substantially, or partially covered with a polymeric covering or coating, but other suitable configurations are contemplated. The covering or coating may extend across and/or occlude one or more, or a plurality of the cells 46 defined by the filaments or struts 36. The covering or coating may help reduce tissue ingrowth and/or may be provided for other suitable reasons.

In a radially expanded configuration, the stent 10 may include a first end region 20 proximate the first end 14 and a second end region 22 proximate the second end 16. In some examples, the first end region 20 and the second end region 22 may include retention features or anti-migration flared regions 24, 26 having enlarged diameters relative to the intermediate region 18. The anti-migration flared regions 24, 26, which may be positioned adjacent to the first end 14 and the second end 16 of the stent 10, may be configured to engage an interior portion of the walls of the biliary tract or other body lumen. In some embodiments, the retention features, or flared regions 24, 26 may have a larger diameter than the cylindrical intermediate region 18 of the stent 10 to prevent the stent 10 from migrating once placed in a body lumen. It is contemplated that transition 28, 30 from the cross-sectional area of the intermediate region 18 to the retention features or flared regions 24, 26 may be gradual, sloped, or occur in an abrupt step-wise manner, as desired.

The first anti-migration flared region 24 may have a first outer diameter and the second anti-migration flared region 26 may have a second outer diameter. In some examples, the first and second outer diameters may be approximately the same, while in other instances, the first and second outer diameters may be different. In some examples, the stent 10 may include only one or none of the anti-migration flared regions 24, 26. In one example, the first end region 20 may include an anti-migration flare region 24 while the second end region 22 may have an outer diameter similar to the intermediate region 18. In another example, the second end region 22 may include an anti-migration flared region 26 while the first end region 20 may have an outer diameter similar to an outer diameter of the intermediate region 18. In some examples, the stent 10 may have a uniform outer diameter from the first end 14 to the second end 16.

The stent 10 may have any suitable diameter. In some examples, the outer diameter of the intermediate region 18 may be in a range of about 1 millimeters (mm) to about 25 mm. The outer diameter of the anti-migration flared regions 24, 26 may be in a range of about 2.0 mm to about 30 mm. In one example configuration of the stent 10 designed for placement in a lumen of the biliary tract, the stent 10 may have an unexpanded diameter equal to or less than 3.0 mm and an expanded outer diameter at the intermediate region 18 of 4.0 mm and 5.0 mm at the flared regions 24, 26. It is contemplated that the outer diameter of the stent 10 may be varied to suit the desired application. It is further contemplated that the stent 10 may include any number of changing features along its length such as, but not limited to, bumps, grooves, ridges, recesses, diameter changes, prongs, etc.

It is contemplated that the elongated tubular member of the stent 10 can be made from a number of one or more different materials. Depending on the material selected for construction, the stent 10 may be self-expanding or require an external force to expand the stent 10. In some configurations, the filaments of the stent 10, or portions thereof, may be bioabsorbable or biodegradable, while in other instances the filaments of the stent 10, or portions thereof, may be biostable

Example materials from which the stent may be formed include, but are not limited to, metals, metal alloys, shape memory alloys, nitinol, cisplatin, polymers, polyethylene terephthalate (PET), and/or other suitable materials enabling the stent 10 to be expanded into shape when accurately positioned within the body. In some instances, the material may be selected to enable the stent 10 to be removed with relative ease as well. For example, the elongated tubular member of the stent 10 can be formed from alloys such as, but not limited to, nitinol, ELGILOY®, and/or other suitable materials. It is further contemplated the elongated tubular member of the stent 10 may be formed from polymers including, but not limited to, polyethylene terephthalate (PET).

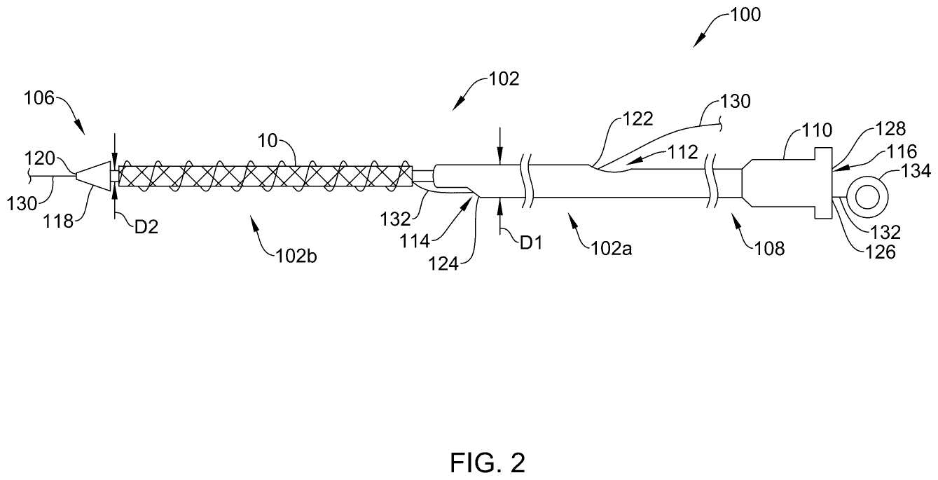

FIG. 2 is a side view of an illustrative delivery system 100 for delivering an implant, such as the stent 10 described herein and/or other suitable stent or implant, to a target region (e.g., a target body lumen). The delivery system 100 may include an elongate shaft 102 (e.g., one or more tubular members). The elongate shaft 102 may extend proximally from a distal end region 106 configured to extend into a patient's body to a proximal end region 108 configured to remain outside of a patient's body. A handle or hub 110 may be coupled to the proximal end region 108 of the elongate shaft 102.

The elongate shaft 102 may include or may be coupled with a distal tip 118 positioned at or proximate to the distal end region 106. The distal tip 118 may be configured to be atraumatic and may taper in a proximal to distal direction, but other suitable configurations of the distal tip 118 are contemplated.

The elongate shaft 102 may include a proximal portion 102a and a distal portion 102b. In some examples, the distal portion 102b may extend from a distal end of the proximal portion 102a to a distal end of the distal tip 118 and the proximal portion 102a may extend from a proximal end of the distal portion 102b to a proximal end of the hub 110, but other suitable configurations are contemplated. In some examples, the proximal portion 102a may terminate at a proximal end that is distal of the proximal end of the hub 110.

The distal portion 102b of the elongate shaft 102 may have a section with a reduced outer diameter relative to the proximal portion 102b. In some examples, the proximal portion 102a of the elongate shaft 102 may extend from the hub 110 to the reduced outer diameter section of the distal portion 102b and the distal portion 102b of the elongate shaft 102 may extend between the distal tip 118 and a distal end of the proximal portion 102a, but other suitable configurations are contemplated. In some examples, the reduce diameter section of the distal portion 102b of the elongate shaft 102 may be spaced distal of a proximal end of the distal portion 102b such that at least a section of the distal portion 102b may have a same or similar outer diameter as the distal end of the proximal portion 102a. The section of the distal portion 102b with a reduced diameter may be configured to receive the stent 10 in a secured manner while maintaining an outer diameter that is less than or equal to a maximum outer diameter of a section of the proximal portion 102a that is configured to extend into the patient.

The elongate shaft 102 may have any suitable outer diameter(s). In some examples, the proximal portion 102a may have any suitable first outer diameter D1 at the section configured to extend into a lumen of the patient and the distal portion 102b may have a second outer diameter D2 at the section with the reduced diameter, where the second outer diameter D2 is less than the first outer diameter D1. Although other suitable outer diameters are contemplated, the first outer diameter D1 may be less than 4 millimeters (mm) (e.g., 12 Fr) or less. In one example, the first outer diameter D1 may be 3 mm (e.g., 9 Fr) or less, but other suitable configurations are contemplated. The second outer diameter D2 may be in a range of about 0.75 mm to about 1.5 mm, in a range of about 1.1 mm to about 1.27, and/or another suitable diameter less than the first outer diameter D1. In some examples, the first outer diameter D1 may be a maximum outer diameter of the elongate shaft or at least a maximum outer diameter of a section of the elongate shaft 102 configured to extend into a patient, but other suitable configurations are contemplated. In some examples, the second outer diameter D2, similar to as discussed above with respect to the reduce diameter section of the distal portion 102b, may be configured to receive the stent 10 in a secured manner while maintaining an outer diameter of the stent 10 that is less than the first outer diameter D1.

The elongate shaft 102 may include one or more lumens. In some examples, the elongate shaft 102 may include a plurality of lumens. In one example, the elongate shaft 102 may include a first lumen 112 extending between a first port 120 (e.g., an opening of or at the first port 120) at the distal tip 118 (e.g., the distal tip 118 may define the opening at the first port 120, but other suitable configurations are contemplated) and a second port 122 (e.g., an opening of or at the second port 122) and a second lumen 114 extending between a third port 124 (e.g., an opening of or at the third port 124) and a fourth port 126 (e.g., an opening of or at the fourth port 126) at or proximate the hub 110. In some examples, the elongate shaft 102 may include a third lumen 116 extending between a fifth port 128 (e.g., an opening of or at the fifth port 128) and a stop (not shown in FIG. 2) at or proximal of the second port 122. The third lumen 116, when included, may be in fluid communication with or fluidly isolated from the first lumen 112 and/or the second port 122. When the third lumen 116 is omitted and/or is not in-line with the first lumen 112, the first lumen 112 may have an opening at the second port 122 and extend proximally to a location at or proximate to the hub 110, but other suitable configurations are contemplated.

The second port 122 may be a side port extending through the elongate shaft 102 from the outer surface to the first lumen 112. In some examples, the second port 122 may be located distal of the hub 110 and proximal of the distal portion 102b of the elongate shaft 102. In one example, the second port 122 may be spaced about 275 mm from the distal tip 118, but the second port 122 may be spaced other suitable distances from the distal tip 118, as desired.

The second port 122 may be any suitable type of port. In one example, the second port 122 may be a skived port (e.g., a first skived port), but other suitable ports are contemplated. When the second port 122 is a skived port and the elongate shaft 102 includes the first lumen 112 and the third lumen 116, the material from the elongate shaft 102 that was skived to form the second port 122 may be utilized to fluidly isolate the first lumen 112 from the third lumen 116.

The first lumen 112 may be configured to receive a guidewire 130. When the second port 122 is a side port, as depicted in FIG. 2, the elongate shaft 102 may be considered to be or may be considered to be part of a rapid exchange catheter, but other suitable configurations are contemplated.

The second lumen 114 may be configured in any suitable manner. In some examples, the second lumen 114 may extend from the third port 124 proximal of the distal portion 102b and/or a distal end of the elongate shaft 102 to the fourth port 126 at or proximate the hub 110. In some examples, the second lumen 114 may be configured to receive a thread 132 (e.g., a filament, a wire, a suture, etc.) with a distal portion securing and/or configured to secure the stent 10 to the elongate shaft 102 (e.g., to the distal portion 102b of the elongate shaft 102). In some examples, the third port 124 at a distal end of the second lumen 114 may be a skived port (e.g., a second skived port) configured to facilitate receiving the thread 132 (e.g., a proximal portion of the thread 132 and/or other suitable portion of the thread 132) and inserting the elongate shaft 102 into a lumen of the body of the patient.

The stent 10 may be disposed around a portion of the distal portion 102b of the elongate shaft 102 at or proximate to the distal end region 106 and proximal of the distal tip 118. When the stent 10 is disposed over the distal portion 102b, in a delivery configuration, the stent 10 may be restrained or contained in a collapsed reduced diameter or delivery configuration, as depicted in FIG. 2, using a containment mechanism in one or more suitable manners. For example, the stent 10 may be restrained or contained in the collapsed reduced diameter or delivery configuration using an outer sheath, one or more wrapped or wound threads, one or more crocheted threads, and/or one or more other suitable containment mechanisms. In some examples, a wrapped, wound, and/or crocheted thread may be considered a sheath.

One or more pieces of threads 132 may be wound, wrapped, and/or crocheted about the distal portion 102b of the elongate shaft 102 and/or an outer surface of the stent 10 to apply a biasing force to the stent 10 which may maintain the stent 10 in the collapsed reduced diameter or delivery configuration. When a force is applied to a proximal end of the thread 132, the thread 132 may unwrap from around the stent 10 to allow the stent 10 to expand (e.g., expand automatically or in response to a user actuation). In some examples, the thread(s) 132 securing the stent 10 in the reduced diameter or delivery configuration may be referred to as a sheath. Utilizing a thread as a stent containment mechanism may mitigate forces needed to release the stent 10 relative to when contain mechanism configured to slide off of the stent 10 due to due radial forces of the stent 10 acting on the containment mechanism as the containment mechanism is slid off of or otherwise over the stent 10.

The thread 132 may be any thin flexible element capable of being wrapped and unwrapped about the stent 10. A distal portion of the thread 132 may be wound, wrapped, or crocheted about the distal portion 102b of the elongate shaft 102 and/or the stent 10 while a proximal portion of the thread 132 may extend proximally through the third port 124, the second lumen 114, and to a location proximal of the hub 110 or other suitable location. In some examples, a proximal end or portion of the thread 132 may be configured to remain proximal of the hub 110 and may be coupled to or otherwise affixed to a pull member or component 134 or other suitable actuation mechanism. Example configurations of the pull component 134 may include, but are not limited to, a pull ring (e.g., as depicted in FIG. 2), a pull tab, a wheel for winding and pulling on the thread 132, and/or other suitable pull components. In some examples, the pull component 134 may be omitted.

The thread 132 may be any suitable material and/or configuration. In some examples, the thread 132 may be or include one or more monofilaments. The cross-section configuration of the suture or filament material may also include any suitable configuration.

Illustrative configurations of the thread 132 wrapped, woven, and/or crocheted for securing a stent 10 or other suitable implant include, but are not limited to, those disclosed in U.S. Pat. Appl. Publ. No. 2010/0331960 and U.S. Pat. Nos. 5,653,748 and 6,019,785, which are each hereby incorporated by reference herein in their entirety for any and all purposes. Other illustrative configurations of the thread 132 configured to secure the stent 10 and/or other implants in the reduced diameter or delivery configuration include, but are not limited to, other patterns and structures including braids, weaves, twists and/or knots.

The thread 132 may be wrapped around the stent 10 in a generally helical manner, although this is not required. The spacing of adjacent windings of the thread 132 may be uniform or variable as desired. For example, the pitch of the thread 132 may be the same, varied, or combinations thereof, as desired. In some cases, the thread 132 may include a plurality of knots similar in form and function to those used in knitting or crocheting, which allow the thread 132 to be releasably secured about the stent 10. The knots may generally maintain the thread 132 in a desired configuration while still allowing the thread 132 to be unraveled or removed as desired. In some examples, the thread 132 may not include knots.

A distal end of the thread 132 may be positioned proximal to the first end region 20 of the stent 10. In other embodiments, the distal end of the thread 132 may be positioned adjacent to the second end region 22 of the stent 10. It is contemplated that the position of the distal end of the thread 132 when the thread 132 is securing the stent 10 in the reduced diameter or delivery configuration may determine which portion of the stent 10 (e.g., proximal or distal) is expanded first. In some examples, the thread 132 may be wound such that the clinician may have the option of selecting which portion of the stent 10 is deployed first.

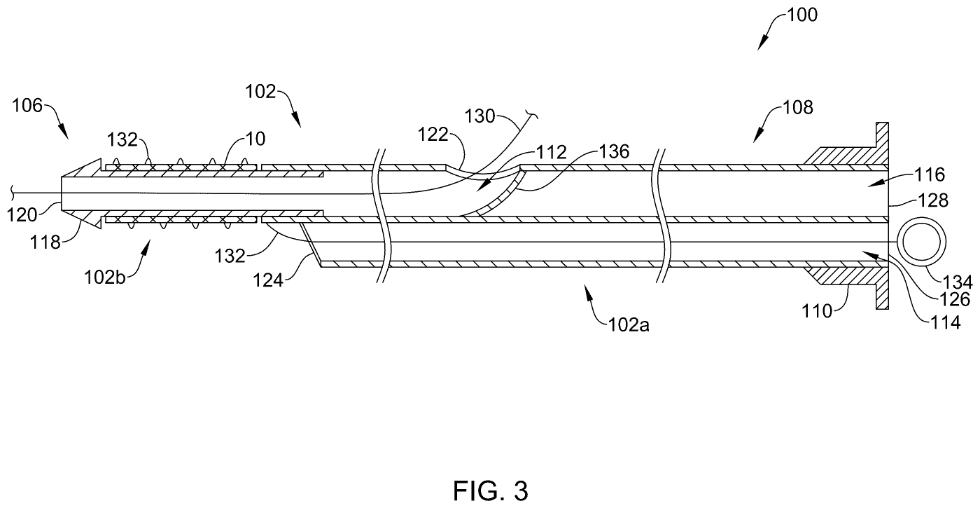

FIG. 3 is a schematic cross-section view of the illustrative delivery system 100 depicted in FIG. 2, with the stent 10 coupled thereto. The delivery system 100 may include one or more lumens extending into and/or through the elongate shaft 102. As depicted in FIG. 3, the delivery system 100 may be or include a rapid exchange catheter configuration with the guidewire 130 extending through the first lumen 112.

The distal portion 102b of the elongate shaft 102 may entirely or at least partially define the first lumen 112, but other suitable configurations are contemplated. In some examples, the first lumen 112 may extend through the distal portion 102b and at least a section of the proximal portion 102a of the elongate shaft 102 between the first port 120 at or proximate the distal tip 118 and the second port 122 extending through a side of the proximal portion 102a of the elongate shaft 102.

The proximal portion 102a of the elongate shaft 102 may entirely or at least partially define the second lumen 114, but other suitable configurations are contemplated. In some examples, the second lumen 114 may extend through the proximal portion 102a of the elongate shaft 102 between the third port 124 located proximal of the first port 120 and distal the second port 122 and the fourth port 126 at or proximate the hub 110. The third port 124 and fourth port 126 may be located at other suitable locations. Although the fourth port 126 is depicted at a proximal end of the hub 110 in FIG. 3, the fourth port 126 may be located at one or more other suitable locations including, but not limited to, a different location along the hub 110 and/or at a location distal of the hub 110 along a section of the elongate shaft 102 that is configured to remain proximal of (e.g., outside of) the patient during a medical procedure in which the delivery system 100 is used.

The proximal portion 102a of the elongate shaft 102 may entirely or at least partially define the third lumen 116, when included, but other suitable configurations are contemplated. In some examples, the third lumen 116, when included, may extend between the fifth port 128 at or proximate the hub 110 and a divider 136 at or proximate the second port 122, as depicted in FIG. 3. In some examples, the third lumen 116 may be configured to receive a stiffening component (e.g., a stiffening shaft, etc.) to facilitate advancing the delivery system 100 through vasculature of the patient and/or may be configured for one or more other suitable purposes. The fifth port 128 and/or the divider 136 may be located at one or more other suitable locations. Although the fifth port 128 is at a proximal end of the hub 110 in FIG. 3, the fifth port 128 may located at one or more other suitable locations including, but not limited to, a different location along the hub 110 and/or at a location distal of the hub 110 along a portion of the elongate shaft 102 that is configured to remain proximal of (e.g., outside of) the patient during a medical procedure in which the delivery system 100 is used.

The divider 136 may have any suitable configuration. In one example, the divider 136 may be material of the elongate shaft 102 that was skived to form the second port 122, which may be affixed to an interior surface of the elongate shaft 102 defining the first lumen 112 and the third lumen 116. When the skived material of the elongate shaft 102 is secured to the interior surface of the elongate shaft 102 to form the divider 136, the divider 136 may fluidly separate the first lumen 112 and the third lumen 116. Other suitable configurations of the divider 136 are contemplated and in some examples, the divider 136 may be omitted such that the delivery system 100 may or may not include the third lumen 116.

The first lumen 112, the second lumen 114, and the third lumen 116 (when included) may have any suitable configuration relative to one another. For example, two or more of the lumens 112, 114, 116 may be parallel to one another, co-axial to one another, serial to or in-line with one another, and/or may be arranged in one or more other suitable manners. In one example, the first lumen 112 and the third lumen 116 may be in-line with one another with a common axis and parallel with the second lumen 114, but other suitable configurations are contemplated. In some examples, the second lumen 114 may overlap with the first lumen 112 and/or the third lumen 116.

The elongate shaft 102 may be formed in any suitable manner. In some examples, the proximal portion 102a of the elongate shaft 102 may be formed from an elongate dual lumen tube (e.g., a first tubular structure) and the distal portion 102b of the elongate shaft 102 may be formed from a single lumen tube (e.g., a second tubular structure) with a proximal end coupled to a distal end of the dual lumen tube. In one example and as depicted in FIG. 3, the proximal end of the single lumen tube may be inserted into a lumen at a distal end of the dual lumen tube and coupled to an interior surface of the lumen in any suitable manner (e.g., adhesive, reflow, etc.) In some examples, the elongate shaft 102 may be formed from a single component (e.g., a monolith configuration) with the plurality of lumens defined in the single component during manufacturing of the elongate shaft 102. In some examples, the elongate shaft 102 may be formed from a plurality of tubes where each lumen of the elongate shaft 102 may be defined by a separate elongate tube and the separate elongate tubes may be coupled together with a further tube and/or other suitable coupling component to form the elongate shaft 102.

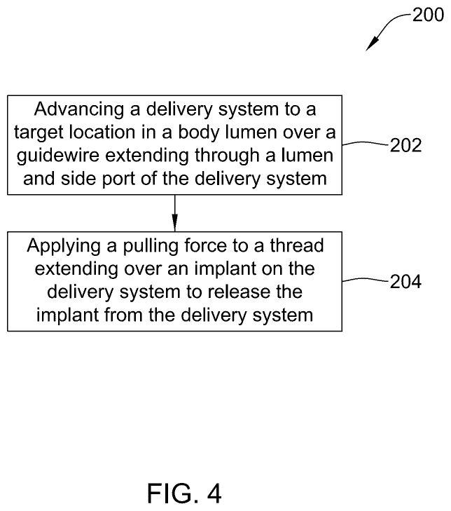

FIG. 4 depicts a schematic box diagram of an illustrative method 200 of delivering an implant (e.g., a stent or other suitable implant) to a target location (e.g., a target body lumen). A delivery system including or having a stent or other suitable implant coupled thereto may be advanced 202 through one or more body lumens to a target body lumen (e.g., a target site in the common bile duct, a target site in any other lumen of the biliary tract, and/or a target site in any other body lumen). The delivery system may be advanced to the target body lumen trans-orally, trans-rectally, and/or in one or more other suitable manners. In some example configurations, a guidewire may be advanced through body lumens to the target body lumen. The delivery system may be advanced to the target body lumen by advancing an elongate shaft of the delivery system over the guidewire, where the guidewire may extend through a first port at a distal tip of the delivery device, through a first lumen of the delivery device, and out of a second port of the delivery device that extends through a side wall of the elongate shaft and is proximal of the stent or implant to be delivered and distal of a hub proximate a proximal end of the elongate shaft. The elongate shaft may be advanced over the guidewire until the stent or other suitable implant, which may be located on a reduced diameter distal portion of the elongate shaft, is positioned at or proximate the target body lumen.

Once stent or other suitable implant coupled with the elongate shaft is positioned at or proximate the target body lumen, a pulling force may be applied 202 to a thread extending over the stent or other suitable implant and restraining or constraining the stent or other suitable implant to the elongate shaft. The pulling force may be applied to the thread so as to unravel the thread from around the stent or other suitable implant and release the stent and/or other suitable implant from the delivery system. In some examples, the pulling force may be applied to a proximal end of the thread until thread is completely unraveled from around the stent or other suitable implant and/or fully or at least partially withdrawn from the delivery system. In some examples, unraveling the thread from the stent or other suitable implant may result in releasing a proximal or distal portion of the stent or implant prior to the other of the distal or proximal portion of the stent or implant, such that one end of the stent or other suitable implant may expand prior to the other end of the stent or other suitable implant, but other suitable configurations are contemplated and all portions of the stent or other suitable implant may be released from the elongate shaft simultaneously.

Once the thread has been removed from covering the stent or other suitable implant, the stent or other suitable implant may expand and secured to the target body lumen (e.g., a target location at a lumen of the biliary tract and/or other suitable location). After releasing the stent or other suitable implant, the delivery device may be removed from the body lumens of the patient.

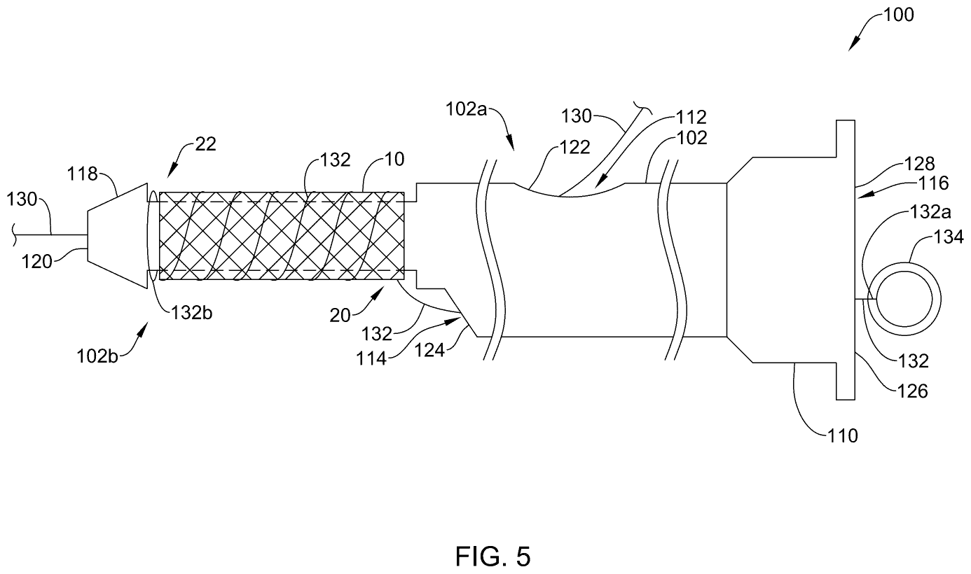

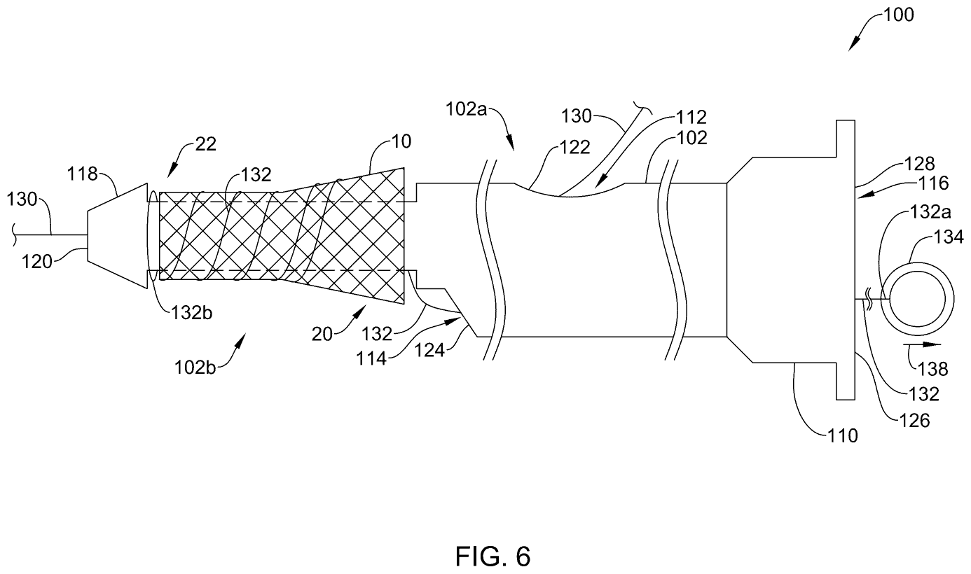

FIGS. 5 and 6 schematically depict an illustrative technique for releasing the stent 10 or other suitable implant in or at a target location of a body lumen using the delivery system 100. FIG. 5 depicts a schematic side view of a distal portion of the delivery system 100 with the stent 10 secured in the collapsed or delivery configuration with the thread 132. The structure of the delivery system 100 may be substantially the same as described above with respect to FIGS. 2 and 3. Once the stent 10 on the reduced diameter section of the distal portion 102b of the elongate shaft 102 is positioned at or proximate the target location, the restraining forces maintaining the stent 10 in the radially collapsed configuration may be removed to deploy the stent 10.

In some example configurations of the thread 132 securing the stent 10 to the elongate shaft 102, the first end region 20 of the stent 10 may be deployed prior to the second end region 22 stent 10. Once the stent 10 is at or proximate to the target location, a proximal or pulling force 138 may be applied to the proximal end 132a of the thread 132, as shown in FIG. 6. In some examples, the pulling force 138 may be applied by placing a finger inside of the pull component 134 and pulling away from the hub 110 and/or by applying the pulling force 138 to a proximal end 132a of thread 132 in one or more other suitable manners. As the pull component 134 and/or the thread 132 is pulled or actuated, the thread 132 around the stent 10 may begin to unravel. In the illustrative configuration shown in FIGS. 5 and 6, the thread 132 may be wound, wrapped, or crocheted around the stent 10 such that the thread 132 disposed over the first end region 20 of the stent 10 is removed or unraveled first. As the biasing force of the thread 132 on the stent 10 is released as the thread 132 is unraveled, the stent 10 may begin to radially expand into its unbiased or deployed configuration, as depicted in FIG. 6. As the thread 132 is pulled, the portion of the thread 132 previously wound around the stent 10 unravels and enters the second lumen 114 at the third port 124 and moves proximally through the second lumen 114. Continued unraveling of the thread 132 may cause more of the length of the stent 10 to be released and expanded. Proximal actuation of the proximal end 132a of the thread 132 may continue until the distal end 132b of the thread 132 has been completely unraveled. It is contemplated that the clinician may continue to pull the thread 132 until the distal end 132b of the thread 132 has been completely removed from the second lumen 114 and the delivery system 100, but other suitable configurations are contemplated.

Once the thread 132 has been fully released and/or separated from the stent 10, the stent 10 may assume its fully deployed configuration, as shown for example in FIG. 1. The delivery system 100 may then be removed from the body lumen.

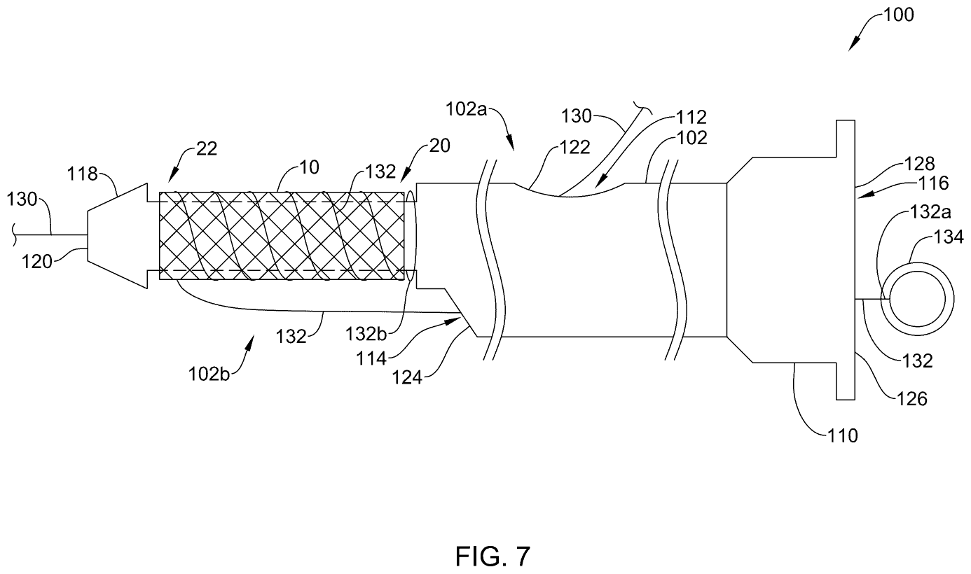

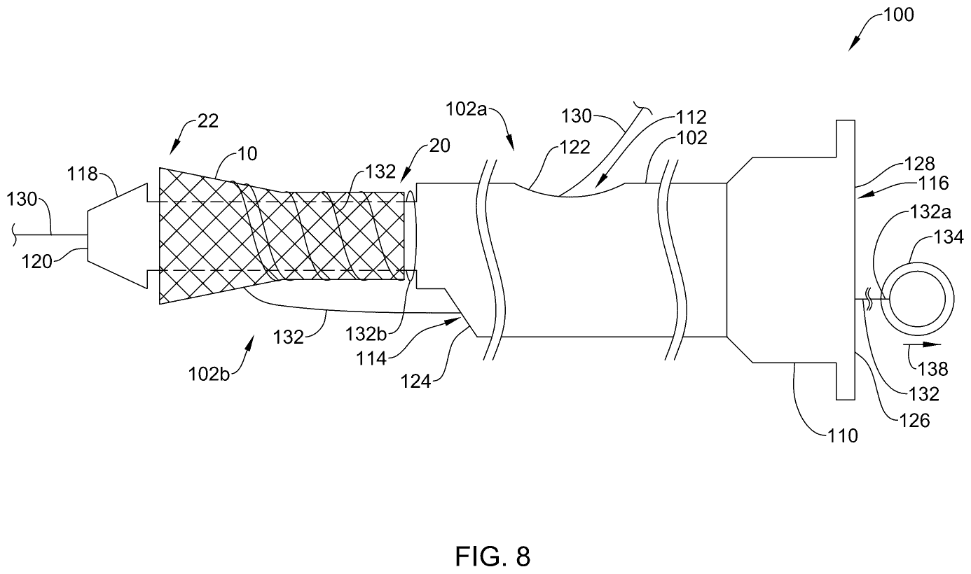

FIGS. 7 and 8 schematically depict another illustrative technique for releasing the stent 10 or other suitable implant in or at a target location of a body lumen using the delivery system 100. The structure of the delivery system 100 may be substantially the same as described above with respect to FIGS. 2 and 3. However, the thread 132 may be wound in a different direction such that the stent 10 may be deployed distally to proximally (as opposed to proximally to distally, as shown in FIGS. 5 and 6).

FIG. 7 depicts a schematic side view of a distal portion of the delivery system 100 with the stent 10 in the collapsed or delivery configuration. The stent 10 may be disposed around the distal portion 102b of the elongate shaft 102. The stent 10 may be held in a radially collapsed configuration through the use of the thread 132. The thread 132 may be wound about an outer surface of the stent 10 to apply a biasing force to the stent 10 which maintains the stent 10 in a collapsed or reduced diameter configuration. In some example configurations, the distal end 132b of the thread 132 may be positioned proximate to the first end region 20 of the stent 10. The thread 132 may be wrapped around the stent 10 in a generally helical manner, although this is not required. The spacing of adjacent windings of the thread 132 may be uniform or variable, as desired. In other words, the pitch of the windings may be the same, varied, or combinations thereof, as desired. In some examples, the thread 132 may include a plurality of knots similar in form and/or function to those used in knitting or crocheting, which allow the thread 132 to be releasably secured about the stent 10. The knots, when included, may generally maintain the thread 132 in a desired configuration while still allowing the thread 132 to be unraveled or removed as desired. In some examples, the thread 132 may not include knots.

The thread 132 may extend through the third port 124 and into the second lumen 114 of the elongate shaft 102. The thread 132 may extend proximally through the second lumen 114 and exit proximally at the hub 110. The proximal end 132a of the thread 132 may be coupled to or otherwise affixed to a pull component 134 and/or other suitable actuation mechanism. The pull component 134 may facilitate actuation of thread 132, however, the pull component 134 or other actuation mechanism may be omitted.

The delivery system 100 may be advanced through one or more body lumens to a target body lumen (e.g., a target site in the common bile duct, a target site in any other lumen of the biliary tract, and/or a target site in any other body lumen). In some examples, the delivery system 100 may be advanced to the target body lumen by advancing the elongate shaft 102 over the guidewire 130 extending through the first port 120 at the distal tip 118 and the second port 122 in the side of the elongate shaft at a location proximal of the stent 10. Once the stent 10 on the reduced diameter section of the distal portion 102b of the elongate shaft 102 is positioned at or proximate the target region, the restraining forces maintaining the stent 10 in the radially collapsed configuration may be removed to deploy the stent 10.

A proximal or pulling force 138 may be applied to the proximal end 132a of the thread 132 to initiate releasing the stent 10 or another suitable implant. In some examples, the pulling force 138 may be applied by placing a finger inside of the pull component 134 and pulling away from the hub 110. As the pull component 134 is pulled or actuated, the thread 132 may begin to unravel. In the example configurations depicted in FIGS. 7 and 8, the thread 132 may be wrapped or wound such that the thread 132 disposed over the second end region 22 of the stent 10 is removed or unraveled first, prior to the thread disposed over the first end region 20 of the stent 10. As the biasing force of the thread 132 is released as the thread 132 is unraveled, the stent 10 may begin to radially expand into its unbiased or deployed configuration. As the thread 132 is pulled, the portion of the thread 132 previously wound around the stent 10 may unravel and enter the second lumen 114 through the third port 124. The thread 132 may then move proximally through the second lumen 114. Continued unraveling of the thread 132 may cause more of the length of the stent 10 to be released. Proximal actuation of the proximal end 132a of the thread 132 may continue until the distal end 132b of the thread 132 has been completely unraveled from around the stent 10. It is contemplated that the clinician may continue to pull the thread 132 until the distal end 132b has been completely removed from the second lumen 114 and the delivery system 100, but this is not required. Once the thread 132 has been removed from around the stent 10, the stent 10 may assume its fully deployed configuration, as shown for example in FIG. 1. The delivery system 100 may then be removed from the body lumen.

The different embodiments of the stent 10 or other suitable implant described here, their mode of operation, etc., are merely representative of the environment in which the present disclosure operates. Accordingly, a variety of alternatively shaped collaborating components may also be used as a substitutive for the purpose of engaging, steering and locating the stent at a desired target site, thus, not limiting the scope of the present disclosure. Further, the disclosed implants may be adequately stretchable, extendable, and retractable, allowing for its flexible deployment. M ore particularly, the configurations described here may be applicable for other medical applications as well, and accordingly, a variety of other medical devices may be used in combination with the implant. Those medical devices may include biopsy forceps, scissors, lithotripters, dilators, other cautery tools, and the like.

Further, while the stent 10 or other suitable implant is generally described, a variety of other configurations and arrangements may also be contemplated and conceived as well. In addition, the operations, devices, and components, described herein may be equally applicable for other purposes where a component is required to be positioned in places where a stricture needs to be addressed or other treatments are desired. Embodiments of the present disclosure are thus applicable to medical and/or non-medical environments. Further, certain aspects of the aforementioned embodiments may be selectively used in collaboration, or removed, during practice, without departing from the scope of the disclosed embodiments.

The present invention may be manifested in a variety of forms other than the specific embodiments described and contemplated herein. Accordingly, departure in form and detail may be made without departing from the scope and spirit of the present disclosure as described in the appended claims.

What is claimed is:

1. A delivery system for delivering an implant to a body lumen, the delivery system comprising:
a hub;
an elongate shaft coupled with the hub, the elongate shaft comprising:
a proximal portion at least partially defining a first lumen and a second lumen;
a distal portion at least partially defining the first lumen; and
a side port in the proximal portion and distal of the hub, the side port is in communication with the first lumen; and
a thread including a distal portion configured to wrap around the distal portion of the elongate shaft and a proximal portion configured to extend through the second lumen.

2. The delivery system of claim 1, wherein the proximal portion of the elongate shaft is formed from a first tubular structure having a first outer diameter and the distal portion of the elongate shaft is formed from a second tubular structure having a second outer diameter smaller than the first outer diameter.

3. The delivery system of claim 2, wherein the first outer diameter is 3.0 millimeters or less.

4. The delivery system of claim 2, wherein the second tubular structure extends into the proximal portion of the elongate shaft.

5. The delivery system of claim 1, wherein the second lumen extends from a location proximate the hub to a distal opening proximal of a distal end of the elongate shaft.

6. The delivery system of claim 5, wherein the side port is a first skived port and the distal opening is a second skived port.

7. The delivery system of claim 1, further comprising:
a pull member coupled to a proximal end of the thread.

8. The delivery system of claim 1, further comprising:
a distal tip at least partially defining the first lumen and forming a distal end of the distal portion of the elongate shaft.

9. The delivery system of claim 8, wherein the distal tip defines an opening in communication with the tip lumen.

10. A delivery system for delivering an implant to a body lumen, the delivery system comprising:
a hub;
an elongate shaft coupled with the hub, the elongate shaft comprising a first lumen, a second lumen, a reduced diameter portion, and a side port in communication with the first lumen and located between the hub and the reduced diameter portion;
an expandable implant disposed about the reduced diameter portion of the elongate shaft; and
a thread including a distal portion wrapped around the expandable implant and a proximal portion configured to extend through the second lumen, wherein the thread is configured to release the expandable implant for expansion in response to pulling on the proximal portion of the thread.

11. The delivery system of claim 10, further comprising:
a distal tip at least partially defining the first lumen and forming a distal end of the distal portion of the elongate shaft.

12. The delivery system of claim 11, further comprising:
a guidewire extending through the distal tip, the first lumen, and the side port.

13. The delivery system of claim 10, wherein the second lumen extends from a location proximate the hub to a distal opening located proximal of the reduced diameter portion of the elongate shaft.

14. The delivery system of claim 10, further comprising:
a pull member coupled to a proximal end of the thread at a location proximate the hub.

15. The delivery system of claim 10, wherein the elongate shaft has a maximum outer diameter of 3.0 millimeters.

16. A method for delivering an implant to a body lumen, the method comprising:
advancing a delivery system to a target location in a body lumen over a guidewire extending through a first lumen and a side port of the delivery system, the delivery system comprising:
a hub;
an elongate shaft coupled with the hub, the elongate shaft comprising the first lumen, a second lumen, a reduced diameter portion, and the side port located distal of the hub;
an expandable implant disposed about the reduced diameter portion of the elongate shaft; and
a thread including a distal portion wrapped around the expandable implant and a proximal portion configured to extend through the second lumen; and
applying a pulling force to the proximal portion of the thread to unravel the thread wrapped around the expandable implant and allow the expandable implant to expand.

17. The method of claim 16, wherein the target location in the body lumen is a target location in a lumen of a biliary tract.

18. The method of claim 16, wherein the expandable implant comprises a self-expanding stent.

19. The method of claim 16, wherein the delivery system comprises a pull member coupled to a proximal end of the thread.

20. The method of claim 16, wherein the elongate shaft has a maximum outer diameter of 3.0 millimeters.
